(19) United States
(12) Patent Application Publication
(10) Pub. No.: US 2004/0077966 A1
(43) Pub. Date: Apr. 22, 2004

(54) Electroencephalogram diagnosis apparatus and method

(75) Inventors: Isao Yamaguchi, Kanagawa (JP); Kazuhisa Ichikawa, Kanagawa (JP)

Correspondence Address: OLIFF & BERRIDGE, PLC, P.O. BOX 19928, ALEXANDRIA, VA, 22320, US

(73) Assignee: Fuji Xerox Co., Ltd., Tokyo (JP)

(21) Appl. No.: 10/418,222

(22) Filed: Apr. 18, 2003

(30) Foreign Application Priority Data
Oct. 17, 2002 (JP) 2002-302610

(51) Int. Cl. A61B 5/04

(52) U.S. Cl. 600/544

(57) ABSTRACT

Measuring electrodes are disposed in positions T5 and T6 according to the international 10-20 system. Electroencephalographic data obtained from these measuring electrodes is received in an input portion, and converted into phase analysis data on a phase plane V-dV/dt by a phase analysis portion. By use of a set of feature parameters selected from an aspect ratio, a V-axis maximum value, a sub/total revolution number ratio and an RL/UB distribution ratio in a feature parameter calculating portion, a Mahalanobis distance is calculated in a Mahalanobis distance calculating portion. The abnormality of the electroencephalogram is judged on the basis of the Mahalanobis distance, and a result of the judgment is outputted.

The present disclosure relates to the subject matter contained in Japanese Patent Application No. 2002-302610 filed on Oct. 17, 2002, which is incorporated herein by reference in its entirety.

### BACKGROUND OF THE INVENTION

1. Field of the Invention

The present invention relates to an electroencephalogram diagnosis technique for automatically diagnosing psychoneurotic disease such as manic-depressive or epilepsy by use of electroencephalographic data.

2. Description of the Related Art

Electroencephalographic diagnosis in the related art is based on visual judgment of a mass of time-series electroencephalographic data by a skilled medical doctor. Thus, there is a problem that the judgment differs from one doctor to another due to their subjectivity, or the work cannot be carried out by any other staff than skilled medical doctors. In addition, for example, as for electroencephalographic data handled for diagnosis of a patient, for example, contracting epilepsy, data gathered for 24 hours has to be analyzed because it cannot be seen when the patient will have a fit. It is therefore necessary to make a diagnosis on a mass of data manually while the patient normally mounted with 16 to 20 electroencephalographic electrodes for a long time is obliged to have a good deal of patience.

### SUMMARY OF THE INVENTION

The present invention is developed in consideration of the foregoing problems, and an object of the invention is to provide an electroencephalogram diagnosis technique in which the burden on a patient in electroencephalogram measurement is reduced, and judgment of electroencephalographic abnormality can be made in a simple and easy way by any other staff than skilled medical doctors.

The present inventor made diligent researches, and developed a method for electroencephalographic diagnosis with a reduced number of electrodes. It has been thought in the related art that electroencephalographic analysis can be made precisely only if a large number of electrodes are brought into contact with a large number of predetermined positions. However, according to the researches of the inventor, the inventors proved that sufficiently useful information for electroencephalographic diagnosis could be obtained by electroencephalogram measurement not with a large number of electrodes in contact with a head portion but with two electrodes.

According to an aspect of the invention, an electroencephalogram is measured from two electrodes located at symmetrical positions as measuring electrodes, and abnormality in the electroencephalogram is discriminated by use of feature parameters obtained by phase space analysis.

As for the two positions of the electrodes, the positions T5 and T6 in the international 10-20system are preferred. Earth electrodes are disposed ear lobes. The earth electrodes for the electrodes at T5 and T6 may be disposed together in one of the ear lobes.

As for the feature parameters to be generated by the phase space analysis, it is preferable to use at least two kinds of feature parameters of (1) an aspect ratio of an electroencephalographic locus on a phase plane V-dV/dt, (2) a maximum value of absolute values of values V on a V-axis on the phase plane V-dV/dt, (3) a ratio of number of sub-revolutions (number of revolutions not including the origin on the phase plane) to total number of revolutions on the phase plane V-dV/dt, and (4) an RL/UB distribution ratio on the phase plane V-dV/dt (a ratio of number of samples of an electroencephalographic locus in two, left and right quadrants to number of samples of the electroencephalographic locus in two, upper and lower quadrants when the phase plane is divided into four, left, right, upper and lower quadrants).

Incidentally, feature parameters obtained in a technique other than the phase space analysis, for example, from the result of frequency analysis, may be used together.

More preferably, only the maximum value of absolute values of values V on the V-axis on the phase plane V-dV/dt, the ratio of the number of sub-revolutions to the total number of revolutions on the phase plane V-dV/dt, and the RL/UB distribution ratio on the phase plane V-dV/dt are selected as the feature parameters.

According to another aspect of the invention, an electroencephalogram diagnosis apparatus includes an input unit, a phase analysis unit, a feature parameter calculating unit, a reference space forming unit, a separation index calculating unit, a judgment unit, an output unit, and inspection electrodes. The input unit inputs time-series electroencephalographic data. The phase analysis unit plots a time derivative dV/dt of cerebral evoked potential V with respect to the cerebral evoked potential V based on the time-series electroencephalographic data to form an electroencephalographic locus on a phase plane V-dV/dt. The feature parameter calculating unit calculates feature parameters on the phase plane V-dV/dt formed by the phase analysis unit. The reference space forming unit forms a reference space using reference learning data concerning the feature parameters. The separation index calculating unit calculates a separation index between the calculated feature parameters and the reference space. The judgment unit judges existence/absence of disease including neurological disease based on the calculated separation index. The output unit outputs existence/absence of disease of a subject based on a judgment result of the judgment unit. The inspection electrodes measures electroencephalogram of the subject number of which is less than ten. Here the “existence/absence of disease” includes possibility for existence/absence of disease as well as real existence/absence of disease. The number of the inspection electrodes may be two.

In this configuration, electroencephalogram diagnosis can be performed while suppressing the burden on a testee. For example, electrodes are disposed in a headphones-type or cap-type wearing device, and electroencephalographic data is supplied to a diagnosis apparatus body by wire or by wireless. A wearing device may be prepared for each electrode, or a wearing device for holding the two electrodes may be prepared.

Incidentally, not only can the invention be implemented as apparatus or a system, but it can be also implemented as a method. In addition, not to say, a part of the invention can be constructed as software. It goes without saying that software products used for making a computer execute such software are also included in the technical scope of the invention.

### BRIEF DESCRIPTION OF THE DRAWINGS

FIG. 1 is a configuration diagram of apparatus showing an embodiment of the invention.

FIG. 2 is a diagram for explaining an example of arrangement of electrodes for use in electroencephalogram measurement.

FIG. 3 is a diagram showing an example of an electroencephalographic locus plotted on a phase plane V-dV/dt.

FIG. 4 is a table showing a list of feature parameters.

FIG. 5 is a chart for explaining comparison between Mahalanobis distances of normal electroencephalographic data and Mahalanobis distances of epileptic electroencephalographic data when 128 feature parameters were used.

FIG. 6 is a table for explaining prime factor feature parameters.

FIG. 7 is a table for explaining the erroneous discrimination ratio when the number of electrodes used was limited.

FIG. 8 is a chart for explaining comparison between Mahalanobis distances of normal electroencephalographic data and Mahalanobis distances of epileptic electroencephalographic data when two measuring electrodes and feature parameters derived from phase space analysis were used.

FIG. 9 is a table for explaining the erroneous discrimination ratio when only one measuring electrode was used.

FIG. 10 is a table for explaining feature parameters used with the one measuring electrode.

FIG. 11 is a table for explaining the erroneous discrimination ratio in each use channel when electrodes T5 and T6 were used.

FIG. 12 is a table for explaining combinations of use channels and feature parameters in FIG. 11.

FIG. 13 is a chart for explaining comparison between Mahalanobis distances of normal electroencephalographic data and Mahalanobis distances of epileptic electroencephalographic data when three specific kinds of feature parameters were used.

### DETAILED DESCRIPTION OF THE PREFERRED EMBODIMENTS

In a first method for calculating feature parameters, the feature parameters are calculated on a phase plane obtained by phase analysis performed on time-series electroencephalographic data. That is, times-series cerebral evoked potential V is plotted on the phase plane V-dV/dt so as to obtain an electroencephalographic locus. Analysis is made on the obtained electroencephalographic locus. A set of intersection points between the V-axis and the electroencephalographic locus is defined as {V₀}, and a set of intersection points between the dV/dt-axis and the electroencephalographic locus is defined as {dV/dt₀}.

In a first method for calculating the aspect ratio, the aspect ratio is calculated using a maximum value |V₀|ₘₐₓ of absolute values of values V in {V₀} and a maximum value |dV/dt₀|ₘₐₓ of absolute values of values dV/dt in {dV/dt₀}, as follows.

$$\frac{{\vert ⅆ V / ⅆ {t}_{0} \vert}_{max}}{{\vert {V}_{0} \vert}_{max}} \qquad ( 1 )$$

In a second method for calculating the aspect ratio, the aspect ratio is calculated using a mean value |V₀|ₘₑₐₙ of absolute values of values V in {V₀} and a mean value |dV/dt₀|ₘₑₐₙ of absolute values of values dV/dt in {dV/dt₀}, as follows.

$$\frac{{\vert ⅆ V / ⅆ {t}_{0} \vert}_{mean}}{{\vert {V}_{0} \vert}_{mean}} \qquad ( 2 )$$

Further, in a third method for calculating the aspect ratio, the aspect ratio is calculated using a variance σ²ᵥₒ of values V in {V₀} and a variance σ²_{dv/dt0} of values dV/dt in {dV/dt₀}, as follows.

$$\frac{{σ}_{dV / {dt}_{0}}^{2}}{{σ}_{{V}_{0}}^{2}} \qquad ( 3 )$$

The V-axis maximum value is a maximum value of absolute values of values V in {V₀}, that is, the following value.

|V₀|max (4)

The method for calculating the ratio of the number of sub-revolutions to the total number of revolutions (sub/total revolution number ratio) will be described below.

The number of revolutions where the electroencephalographic locus is prevented from including the origin inside on the phase plane V-dV/dt is defined as the number of sub-revolutions N_{sub}. On the other hand, the number of revolutions regardless of whether the electroencephalographic locus includes the origin or not is defined as the total number of revolutions Nₐₗₗ. At this time, the sub/total revolution number ratio is calculated by:

$$\frac{{N}_{sub}}{{N}_{all}} \qquad ( 5 )$$

Next, the method for calculating the RL/UB distribution ratio will be described below.

The axis obtained by rotating the V-axis counterclockwise at an angle of 45° is defined as V′-axis, and the axis obtained by rotating the dV/dt-axis counterclockwise at an angle of 45° is defined as (dV/dt) ′-axis. Four areas on the phase plane divided by these two axes are defined as follows.

When any point on the phase plane is expressed by (x, Y),

| | |
|---|---|
| U area: | y ≧ x, y > −x |
| B area: | y ≦ x, y < −x |
| R area: | y < x, y ≧ −x |
| L area: | y > x, y ≦ −x |

In addition, here, sampling is carried out upon the electroencephalographic locus on the phase plane so as to regard the electroencephalographic locus as a set of points on the phase plane.

At this time, the method for calculating the RL/UB distribution ratio is expressed by:

$$\frac{\begin{matrix}( number of sampled points in R area ) + \\ ( number of sampled points in L area )\end{matrix}}{\begin{matrix}( number of sampled points in U area ) + \\ ( number of sampled points in B area )\end{matrix}} \qquad ( 6 )$$

In addition, in the embodiment of the invention, the Mahalanobis-Taguchi System method (hereinafter referred to as “MTS method”) is used as the method for judging the existence/absence of psychoneurotic disease. The MTS method is a method in which with data, which is classified by human, provided as learning data, a correlation among feature parameters inherent in this learning data set is extracted so that a virtual reference data space reflecting the human ability of discrimination can be generated, and pattern recognition is performed on the basis of a Mahalanobis distance from this reference data space. Also, the method has such a feature that by giving noise to the learning data, discrimination with robustness can be attained. Furthermore, the feature parameters are optimized from the result of the discrimination so that any effective feature parameter can be extracted again. If requiring the details of the MTS method, see “Mathematical Principles of Quality Engineering” by Genichi Taguchi, Quality Engineering Vol. 6No. 6by Quality Engineering Society, pp.5-10 (1998), the entire contents of this reference incorporated herein by reference.

In the discrimination based on the MTS method, a reference data space is generated from a set of learning data, and whether unknown data belongs to the reference data space or not is judged based on its Mahalanobis distance from the generated reference data space.

The reference data space is generated in the following procedure.

[Step 1]:

Normalization of a learning data set: When the number of feature parameters of the learning data is k, the number of elements of the set of learning data is n, and value of each of learning data is xᵢⱼ (i=1, . . . , n, j=1, . . . , k), the learning data set is converted by the following expression using the mean value mⱼ and the standard deviation σⱼ of the learning data set so as to calculate Xᵢⱼ.

$${X}_{ij} = \frac{{x}_{ij} - {m}_{j}}{{σ}_{j}} ( i = 1 , ⋯ , n ; j = 1 , ⋯ , k ) \qquad ( 7 )$$

[Step 2]:

Calculation of correlation matrix: A correlation matrix R is calculated from the normalized learning data set.

$$\begin{matrix}R = [ \begin{matrix}1 & {r}_{12} & ⋯ & {r}_{1 k} \\ {r}_{21} & 1 & ⋯ & {r}_{2 k} \\ ⋮ & ⋮ & ⋰ & ⋮ \\ {r}_{k1} & {r}_{k2} & ⋯ & 1\end{matrix} ] \\ {r}_{ij} = \frac{1}{n} {\sum }_{l = 1}^{n} {X}_{li} {X}_{ij} ( i , j = 1 , ⋯ , k )\end{matrix} \qquad ( 8 )$$

[Step 3]

Calculation of inverse matrix: An inverse matrix A of the correlation matrix R is calculated.

$$A = {R}^{- 1} = [ \begin{matrix}{a}_{11} & {a}_{12} & ⋯ & {a}_{1 k} \\ {a}_{21} & {a}_{22} & ⋯ & {a}_{2 k} \\ ⋮ & ⋮ & ⋰ & ⋮ \\ {a}_{k1} & {a}_{k2} & ⋯ & {a}_{kk}\end{matrix} ] \qquad ( 9 )$$

The mean value mⱼ and the standard deviation σⱼ, and the inverse matrix A of the correlation matrix R are used as a reference space pattern.

In the embodiment of the invention, the physical quantity of a scalar indicating the distance from the reference data space is defined as a separation index. In the embodiment of the invention, a Mahalanobis distance is used for calculating the separation index. The Mahalanobis distance can be regarded as “distance in consideration of correlation” among feature parameters, in comparison with a Euclidean distance used generally. By use of the Mahalanobis distance, it can be judged whether the subject of discrimination belongs to the reference data space pattern or not.

The Mahalanobis distance of a subject of discrimination y (the number of feature parameters is k) can be calculated in the following manner.

The Mahalanobis distance D² is calculated by the following expression using a normalized value Y of the subject of discrimination y on the basis of the mean value mⱼ and the standard deviation σⱼ of the learning data set, which are calculated when the reference space is generated.

$$\begin{matrix}Y = \{ {Y}_{1} , {Y}_{2} , ⋯ , {Y}_{k} \} \\ {D}^{2} = \frac{{Y}^{T} AY}{k}\end{matrix} \qquad ( 10 )$$

In addition, the procedure for analyzing prime factors of the respective feature parameters is defined in the MTS method. By analyzing the prime factors, feature parameters effective for discrimination can be extracted. The procedure for analyzing the prime factors is as follows.

[Step 1]:

Each feature parameter is allocated on an orthogonal array.

[Step 2]:

A reference space based on the orthogonal array is reproduced.

[Step 3: Calculation of SN Ratio]:

An SN ratio is calculated based on the calculated Mahalanobis distance. The SN ratio is an index indicating the separation between the reference space and a sample to be discriminated. The increase of the SN ratio shows that data samples not belonging to the reference space can be discriminated accurately. In the embodiment of the invention, the SN ration is defined as follows.

$$η = - 10 log \frac{1}{d} ( \frac{1}{{D}_{1}^{2}} + \frac{1}{{D}_{2}^{2}} + ⋯ + \frac{1}{{D}_{d}^{2}} ) \qquad ( 11 )$$

η:SN ratio

d:number of data samples not belonging to reference space used for prime factor analysis

[Step 4: Evaluation of Feature Parameters]:

The SN ratio when each feature parameter is used and the SN ratio when the feature parameter is not used are calculated so that a factor effect chart is created.

[Step 5: Selection of Feature Parameters]:

Feature parameters each providing an SN ratio reduced when it is used, that is, feature parameters each having a small factor effect are deleted on the basis of the factor effect chart.

In the embodiment of the invention, a measurement electrode for abnormal electroencephalogram judgment and a feature parameter are determined using this prime factor analysis.

(Embodiment)

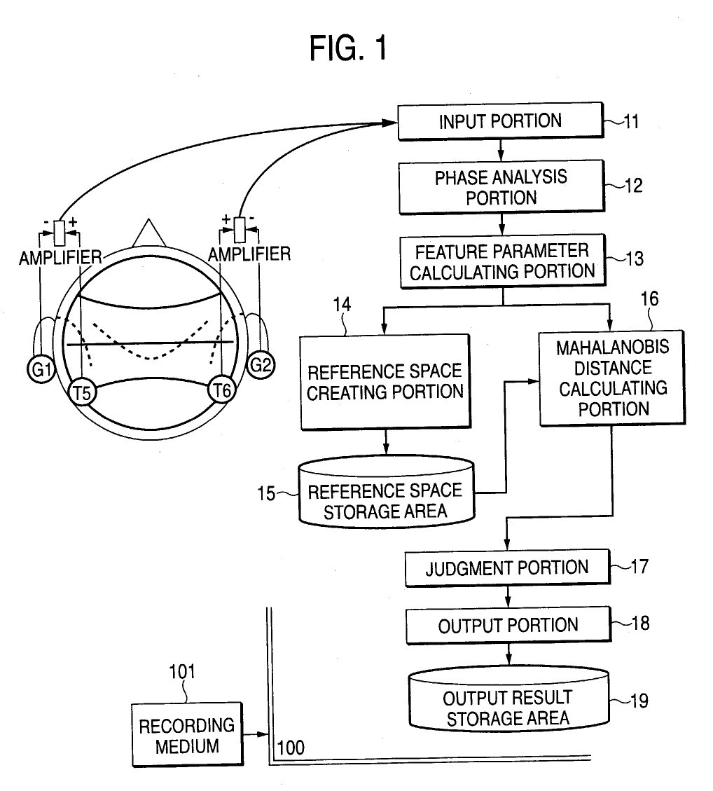

An embodiment of the invention will be described below in detail with reference to the drawings. FIG. 1 is a block diagram showing electroencephalogram diagnosis apparatus (electroencephalogram analyzer) according to an embodiment of the invention.

Cerebral evoked potential obtained from two channels between a measuring electrode T5 and its reference electrode G1 and between a measuring electrode T6 and its reference electrode G2 is supplied to an input portion 11 (the names of the respective electrodes conform to the international 10-20 system). The input data at this time is time-series data of cerebral evoked potential.

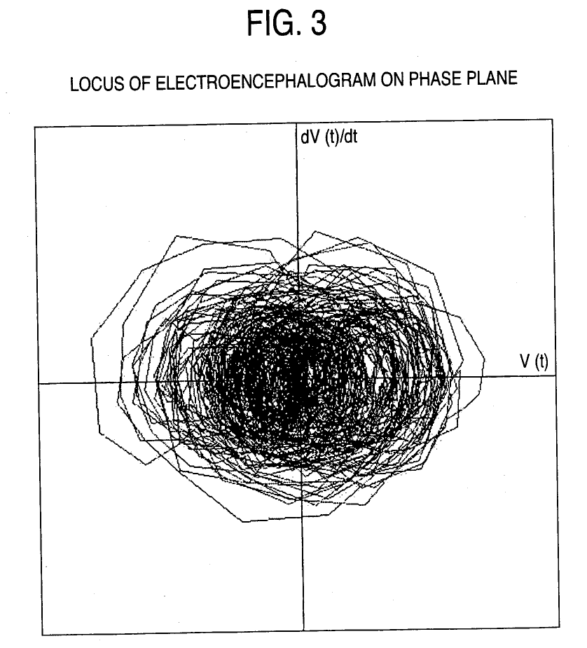

A phase analysis portion 12 in FIG. 1 converts the two-channel input time-series data into an electroencephalographic locus on a phase space. An example of the electroencephalographic locus is shown in FIG. 3. A feature parameter calculating portion 13 calculates feature parameters from the electroencephalographic locus obtained by the phase analysis portion 12.

To create a reference space using a reference learning electroencephalographic data set, which is used in judgment of abnormal electroencephalograms, a reference space creating portion 14 calculates a mean, a variance, and an inverse matrix of a correlation matrix of the reference learning electroencephalographic data set in accordance with the feature parameters calculated by the feature parameter calculating portion 13 and Expressions 7-9, and stores them into a reference space storage area 15 as a reference space.

For judging the existence/absence of abnormality in an electroencephalogram, a Mahalanobis distance calculating portion 16 obtains a Mahalanobis distance in accordance with Expression 10 from the mean, the variance, and the inverse matrix of the correlation matrix of the reference learning electroencephalographic data set calculated as a reference space, and the feature parameters calculated form the electroencephalographic data to be discriminated.

A judgment portion 17 judges normality/abnormality of the discrimination-target electroencephalogram in accordance with the Mahalanobis distance. The judgment result is stored in an output result storage area 19 by an output portion 18.

In this embodiment, by use of the two measuring electrodes T5 and T6, the discrimination of an abnormal electroencephalogram is made using, as feature parameters, an aspect ratio, a V-axis maximum value, a sub/total revolution number ratio and an RL/UB distribution ratio derived from phase space analysis.

The electroencephalogram diagnosis apparatus according to this embodiment can be implemented by a computer 100 such as a personal computer. For example, an electroencephalogram diagnosis program is installed in the computer system 100 through a recording medium 101 or a communication unit.

It will be proved below that by use of the two measuring electrodes T5 and T6, abnormal electroencephalograms can be discriminated precisely using, as feature parameters, the aspect ratio, the v-axis maximum value, the sub/total revolution number ratio and the RL/UB distribution ratio derived from phase space analysis.

The feature parameter calculating portion 13 calculates the aspect ratio, the V-axis maximum value, the sub/total revolution number ratio and the RL/UB distribution ratio in accordance with Expressions 1-7. A deviation of the distribution of histograms of the number of times of crossing on the V-axis, and the ratio of the number of samples in a right quadrant to the number of samples in a left quadrant in the phase space (hereinafter referred to as “RL distribution ratio”) were used as well as the feature parameters. Further, a peak frequency and a ratio of a peak spectrum to a second peak spectrum (hereinafter also referred to as “spectrum ratio”) obtained by Fourier analysis were also used.

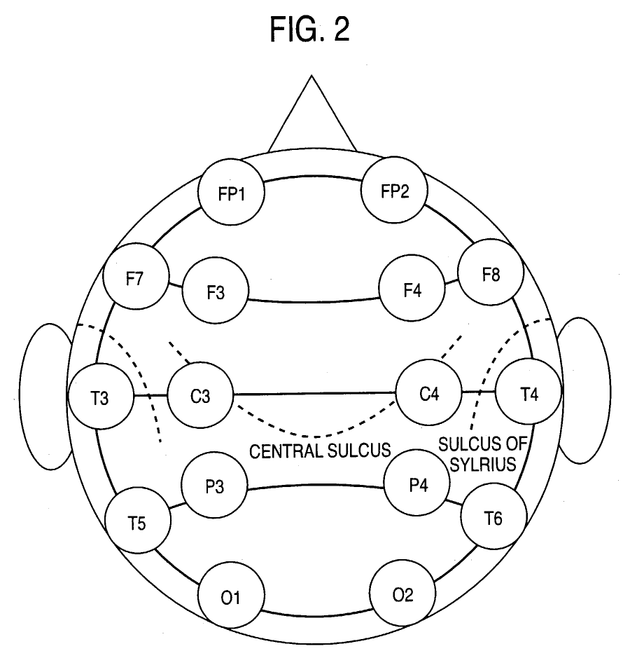

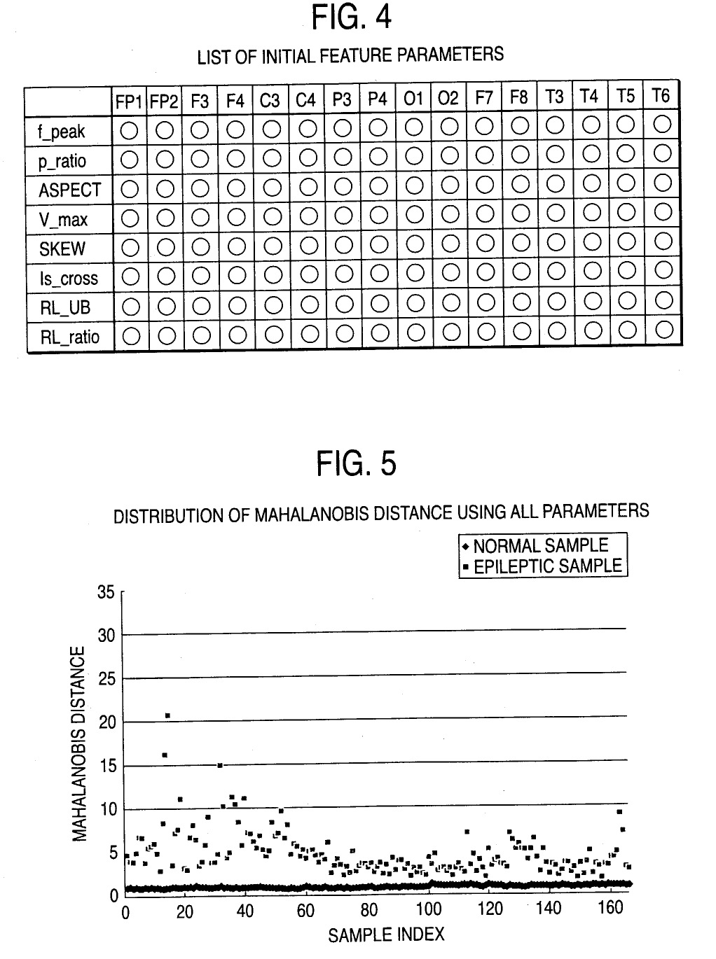

First, to decide good positions of the measuring electrodes and good feature parameters, measuring was performed using measuring electrodes at 16 points shown in FIG. 2. As for the number of feature parameters including the measuring points, there are a total of 128 feature parameters as shown in FIG. 4. Of the feature parameters shown in FIG. 4, “aspect” designates the aspect ratio; “V_max”, the V-axis maximum value; “ls_cross”, the sub/total revolution number ratio; “RL_UB”, the RL/UB distribution ratio; “f_peak”, the value of the peak frequency obtained by Fourier analysis; “p_ratio, the spectrum ratio; “skew”, a deviation of the distribution of histograms of the number of times of crossing on the V-axis; and “RL_ratio”, the RL distribution ratio.

The spectrum ratio “p_ratio” is expressed by:

$$\frac{{F}_{2}}{{F}_{1}} \qquad ( 12 )$$

where F₁ designates the peak value of the spectrum, and F₂ designates the second peak value of the spectrum to the peak value F₁.

The deviation “skew” in the distribution of histograms of the number of times of crossing on the V-axis is expressed using a normal distribution N (x) obtained using histograms H (x) of {V₀}, the mean V₀ₘₑₐₙ and the variance σ²ᵥ₀ of values V in {V₀}, as follows.

$${\sum }_{x \geq 0} \frac{H ( x ) - N ( x )}{N ( 0 )} - {\sum }_{x < 0} \frac{H ( x ) - N ( x )}{N ( 0 )} \qquad ( 13 )$$

Directly using the definitions used for describing the method for calculating the RL/UB distribution ratio, the RL distribution ratio “RL_ratio” is expressed by:

$$\frac{( numberofsampledpointsinRarea )}{( numberofsampledpointsinLarea )} \qquad ( 14 )$$

As the reference learning electroencephalographic data set, 191 samples of normal 10-second electroencephalographic data were prepared, and a reference space for the normal condition was created based on the samples.

The Mahalanobis distances of 166 samples of epileptic data and the Mahalanobis distances of 166 samples of the normal electroencephalographic data used for creating the reference space are shown in FIG. 5. It is understood that the normal electroencephalographic data and the epilepsy electroencephalographic data are separated. The average Mahalanobis distance of the normal electroencephalographic samples was 0.99 while the average Mahalanobis distance of the epileptic samples was 4.78. It is understood that abnormal electroencephalograms can be discriminated in the state where all the channels and all the feature parameters are used. However, in this state, 16 measuring electrodes and 2 reference electrodes, that is, a total of 18 electrodes are required.

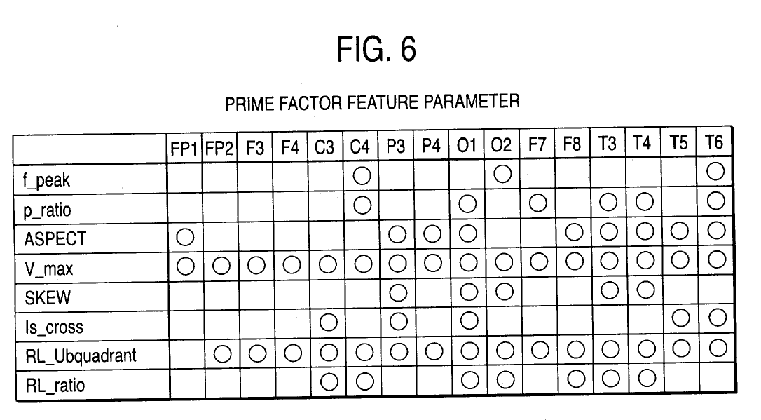

Next, using the 166 samples of epileptic data, prime factor analysis using the prime factor analysis method was performed. As a result, prime factor feature parameters were obtained as shown in FIG. 6. The channels to be used were narrowed down on the basis of the result of FIG. 6. Then, in consideration of the number of prime factor feature parameters and the easiness to dispose the measuring electrodes, the following three sets were aimed at.

O1/O2

T3/T4

T5/T6

It was examined whether abnormal electroencephalograms could be discriminated or not respectively when a reference space was created using two sets of these sets and when a reference space was created using only one set of them. On this occasion, on the assumption that the number of normal electroencephalographic samples each having a larger Mahalanobis distance than a minimum Mahalanobis distance (D_{A,min}) of abnormal electroencephalograms was n_{A} in each condition, and the ratio n_{A}/N of n_{A} to the total number N (=191) of normal electroencephalographic samples was an erroneous discrimination ratio R, 5% or lower in the erroneous discrimination ratio R was established as a threshold for practical use. This reason is as follows. It is allowable that a normal electroencephalogram is judged as an abnormal electroencephalogram because the electroencephalogram can be verified by a medical doctor. However, the reverse erroneous discrimination cannot be allowed. Thus, the threshold value for 100% discriminating abnormal electroencephalograms calculated using a reference space in each condition has to be set to be lower than the minimum Mahalanobis distance D_{A,min}.

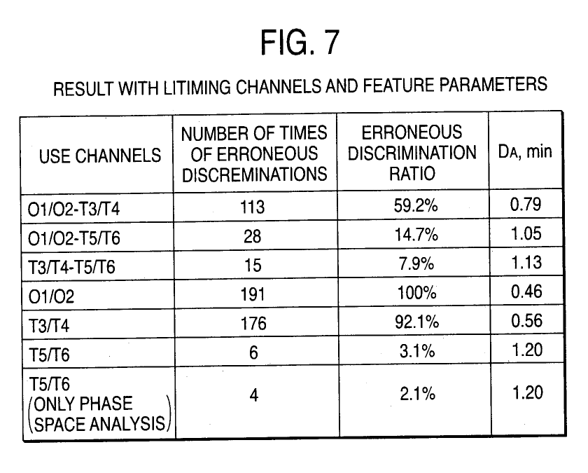

FIG. 7 shows each set of channels used, a corresponding erroneous discrimination ratio R and a corresponding minimum Mahalanobis distance D_{A,min}. In FIG. 7, when the two channels T5 and T6 were used, the erroneous discrimination ratio was 3.1%, which was below the threshold 5%. Thus, this set was judged to be able to be put into practical use.

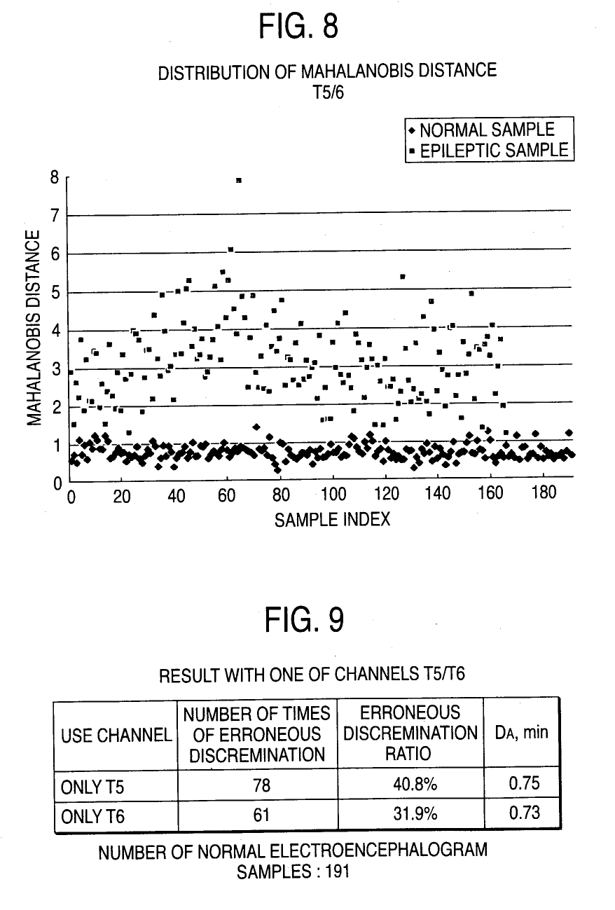

Further, the lowest line of FIG. 7 shows the result using the channels T5 and T6 and only four kinds of feature parameters derived from phase space analysis. The erroneous discrimination ratio is 2.1%, which is further smaller than 3.1% in the result using the feature parameters derived from Fourier analysis. FIG. 8 shows the distribution of Mahalanobis distances of the normal electroencephalographic samples and that of the epileptic electroencephalographic samples at this time.

From above, it was proved that by use of the two measuring electrodes T5 and T6, an abnormal electroencephalogram could be discriminated precisely using, as feature parameters, the aspect ratio, the v-axis maximum value, the sub/total revolution number ratio and the RL/UB distribution ratio derived from phase space analysis.

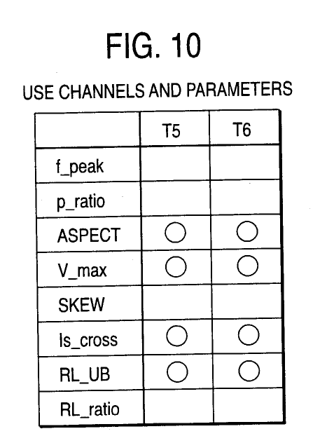

Incidentally, it will be more convenient for only one of the electrodes T5 and T6 to lead to precise electroencephalogram diagnosis. However, precise electroencephalogram diagnosis could not be made satisfactorily with only one of the electrodes T5 and T6, as shown in FIG. 9. In this example, feature parameters shown in FIG. 10 were used. It is therefore understood that it is optimal for simple and precise electroencephalogram diagnosis to use the two electrodes.

Incidentally, the invention is not limited to the embodiment, but various modifications can be made thereon without departing the gist of the invention. For example, although the embodiment has shown the case where the aspect ratio, the v-axis maximum value, the sub/total revolution number ratio and the RL/UB distribution ratio derived from phase space analysis were used as feature parameters while the two measuring electrodes T5 and T6 were used, a set of the V-axis maximum value, the sub/total revolution number ratio and the RL/UB distribution ratio may be used as feature parameters.

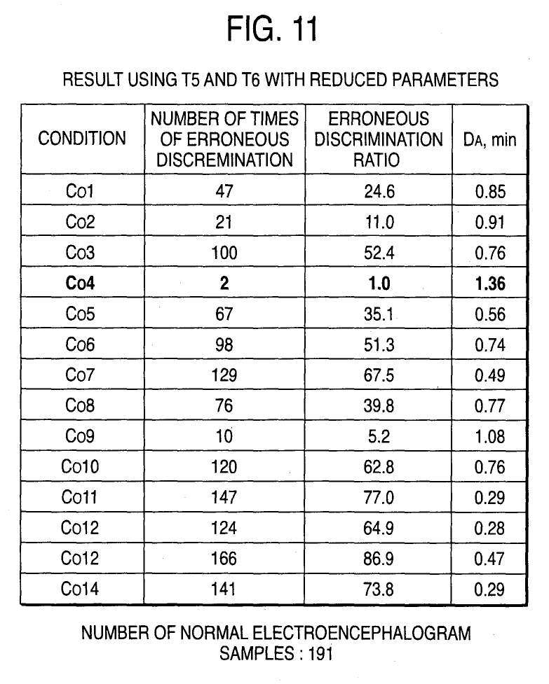

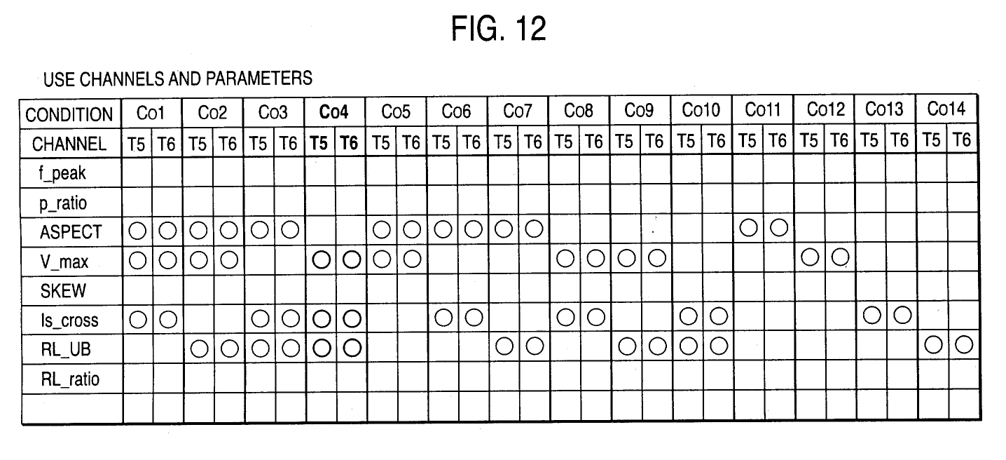

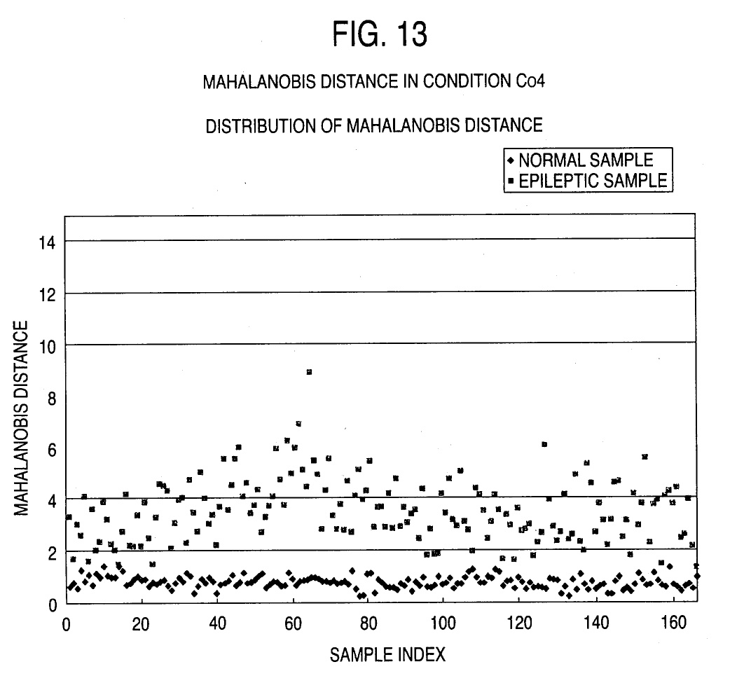

FIG. 12 shows combinations of use channels and one kind of feature parameter or two or three kinds of feature parameters. FIG. 11 shows erroneous discrimination ratios under the conditions of a variety of such combinations (14 conditions Co1 to Co14). In the condition Co4, that is, when the V-axis maximum value, the sub/total revolution number ratio and the RL/UB distribution ratio were used, the erroneous discrimination ratio of 1% could be attained. FIG. 13 shows the distribution of Mahalanobis distances of the normal electroencephalographic samples and the distribution of Mahalanobis distances of the epileptic electroencephalographic samples at that time.

As is apparent from the above description, according to this embodiment, for example, the two points T5 and T6 are adopted as measuring electrodes, and the aspect ratio, the v-axis maximum value, the sub/total revolution number ratio and the RL/UB distribution ratio derived from phase space analysis are selectively used as feature parameters. Thus, abnormal electroencephalograms can be discriminated. According to this embodiment, not a large number of electrodes required for electroencephalogram measurement in the related art but only two measuring electrodes are used. Thus, abnormal electroencephalograms can be discriminated while the burden on a patient is reduced on a large scale and the burden on an operating staff is reduced.

Through this embodiment, it is proved by use of the electrodes T5 and T6 that the number of those measuring electrodes is minimal, and a proper result can be obtained by the number of electrodes. However, according to the invention, it is also possible to add measuring electrodes other than the electrodes T5 and T6. On that occasion, the positions of the added measuring electrodes used conform to those in the international 10-20 system, but the number of measuring electrodes is set to be smaller than the number of electrodes used in the international 10-20 system, specifically to be smaller than 10. Also in this case, there is an effect that the burden on a testee can be reduced.

As is apparent from the above description, according to the invention, abnormal electroencephalograms can be discriminated precisely. In addition, the burden on a patient can be reduced, and the burden on an operating staff can be also reduced.

What is claimed is:

1. An electroencephalogram diagnosis apparatus comprising:
an input unit for inputting time-series electroencephalographic data;
a phase analysis unit for plotting a time derivative dV/dt of cerebral evoked potential V with respect to the cerebral evoked potential V based on the time-series electroencephalographic data to form an electroencephalographic locus on a phase plane V-dV/dt;
a feature parameter calculating unit for calculating feature parameters on the phase plane V-dV/dt formed by the phase analysis unit;
a reference space forming unit for forming a reference space using reference learning data concerning the feature parameters;
a separation index calculating unit for calculating a separation index between the calculated feature parameters and the reference space;
a judgment unit for judging existence/absence of disease including neurological disease based on the calculated separation index;
an output unit for outputting existence/absence of disease of a subject based on a judgment result of the judgment unit; and
inspection electrodes for measuring electroencephalogram of the subject number of which is less than ten.

2. The electroencephalogram diagnosis apparatus according to claim 1, wherein the inspection electrodes are disposed at least at T5 and T6 in international 10-20 system.

3. The electroencephalogram diagnosis apparatus according to claim 1, wherein the number of the inspection electrodes is two.

4. The electroencephalogram diagnosis apparatus according to claim 3, wherein the inspection electrodes are disposed at T5 and T6 in international 10-20 system.

5. The electroencephalogram diagnosis apparatus according to claim 3, wherein the feature parameter calculating unit calculates an aspect ratio of an electroencephalographic locus on the phase plane V-dV/dt as the feature parameters.

6. The electroencephalogram diagnosis apparatus according to claim 3, wherein the feature parameter calculating unit calculates a maximum value of absolute values of V on a V-axis on the phase plane V-dV/dt as the feature parameters.

7. The electroencephalogram diagnosis apparatus according to claim 3, wherein the feature parameter calculating unit calculates a ratio of number of sub-revolutions to total number of revolutions on the phase plane V-dV/dt as the feature parameters.

8. The electroencephalogram diagnosis apparatus according to claim 3, wherein the feature parameter calculating unit calculates an RL/UB distribution ratio on the phase plane V-dV/dt as the feature parameters.

9. The electroencephalogram diagnosis apparatus according to claim 3, wherein the feature parameter calculating unit calculates a maximum value of absolute values of values V on a V-axis on the phase plane V-dV/dt, a ratio of number of sub-revolutions to total number of revolutions on the phase plane V-dV/dt, and an RL/UB distribution ratio on the phase plane V-dV/dt as the feature parameters.

10. The electroencephalogram diagnosis apparatus according to claim 5, wherein the aspect ratio is a ratio of a maximum value of absolute values in a first histogram of intersection points between a V-axis of the phase plane V-dV/dt and the electroencephalographic locus, to a maximum value of absolute values in a second histogram of intersection points between a dV/dt-axis of the phase plane V-dV/dt and the electroencephalographic locus.

11. The electroencephalogram diagnosis apparatus according to claim 5, wherein the aspect ratio is a ratio of a mean vale of absolute values in a first histogram of intersection points between a V-axis of the phase plane V-dV/dt and the electroencephalographic locus, to a mean value of absolute values in a second histogram of intersection points between a dV/dt-axis of the phase plane V-dV/dt and the electroencephalographic locus.

12. The electroencephalogram diagnosis apparatus according to claim 5, wherein the aspect ratio is a ratio of a variance of absolute values in a first histogram of intersection points between a V-axis of the phase plane V-dV/dt and the electroencephalographic locus, to a variance of absolute values in a second histogram of intersection points between a dV/dt-axis of the phase plane V-dV/dt and the electroencephalographic locus.

13. The electroencephalogram diagnosis apparatus according to claim 3, wherein a variance, a mean and an inverse matrix of a correlation matrix of the feature parameters in the reference learning data are used as the reference space.

14. The electroencephalogram diagnosis apparatus according to claim 3, wherein a Mahalanobis distance is used as the separation index between the feature parameters and the reference space.

15. An electroencephalogram diagnosis method comprising:
measuring electroencephalographic data of a subject with less than ten inspection electrodes;
inputting time-series electroencephalographic data;
plotting a time derivative dV/dt of cerebral evoked potential V with respect to the cerebral evoked potential V based on the time-series electroencephalographic data to form an electroencephalographic locus on a phase plane V-dV/dt;
calculating feature parameters on the phase plane V-dV/dt formed by the plotting step;
forming a reference space using reference learning data concerning the feature parameters;
calculating a separation index between the calculated feature parameters and the reference space;
judging existence/absence of disease including neurological disease based on the calculated separation index; and
outputting existence/absence of disease of the subject based on a judgment result of the judging step.

16. The electroencephalogram diagnosis method according to claim 15, wherein the inspection electrodes are disposed at least at T5 and T6 in international 10-20 system.

17. The electroencephalogram diagnosis method according to claim 15, wherein the number of the inspection electrodes is two.

18. A recording medium readable by a computer and recording an electroencephalogram computer diagnosis program making the computer execute:
inputting time-series electroencephalographic data using less than ten inspection electrodes;
plotting a time derivative dV/dt of cerebral evoked potential V with respect to the cerebral evoked potential V based on the time-series electroencephalographic data to form an electroencephalographic locus on a phase plane V-dV/dt;
calculating feature parameters on the phase plane V-dV/dt formed by the plotting step;
forming a reference space using reference learning data concerning the feature parameters;
calculating a separation index between the calculated feature parameters and the reference space;
judging existence/absence of disease including neurological disease based on the calculated separation index; and
outputting existence/absence of disease of the subject based on a judgment result of the judging step.

19. The recording medium according to claim 18, wherein the inspection electrodes are disposed at least at T5 and T6 in international 10-20 system.

20. The recording medium according to claim 18, wherein the number of the inspection electrodes is two.
